(19) United States
(12) Patent Application Publication
(10) Pub. No.: US 2010/0241240 A1
(43) Pub. Date: Sep. 23, 2010

(54) SELF-ADJUSTING ENDOURETHRAL DEVICE & METHODS OF USE

(75) Inventors: Lloyd K. Willard, Miltona, MN (US); Mark J. Whalen, Alexandria, MN (US); John M. Reid, Garfield, MN (US); Chad S. Smith, Parkers Prairie, MN (US); Charles L. Euteneuer, St. Michael, MN (US)

Correspondence Address: NAWROCKI, ROONEY & SIVERTSON;SUITE 401, BROADWAY PLACE EAST, 3433 BROADWAY STREET NORTHEAST, MINNEAPOLIS, MN, 554133009, US

(73) Assignee: ABBEYMOOR MEDICAL, INC., Parkers Prairie, MN (US)

(21) Appl. No.: 11/993,411

(22) PCT Filed: Jun. 20, 2006
(86) PCT No.: PCT/US06/23805

§ 371 Date: Jun. 9, 2010

Related U.S. Application Data
(60) Provisional application No. 60/691,636, filed on Jun. 20, 2005.
(60) Provisional application No. 60/691,635, filed on Jun. 20, 2005.

(51) Int. Cl. A61F 2/04

(52) U.S. Cl. 623/23.66

(57) ABSTRACT

A self adjusting and/or positioning indwelling urethral device is provided. The device generally includes a prostatic urethral stent body and a urethral anchoring element. The prostatic urethral stent body includes a preconfigured end portion for anchored receipt within a bladder with the urethral anchoring element extending from the prostatic urethral stent body via a linkage. Engagement of the proximal anchor, delimited by the preconfigured end portion of the stent body, with the bladder is no or minimal impact upon the trigone region.

This is an international application filed under 35 USC §363, claiming priority under 35 USC §119(e) of U.S. Prov. Appl. Nos. 60/691,635 and 60/691,636, each filed Jun. 20, 2005 and each incorporated herein by reference in its entirety.

### TECHNICAL FIELD

The present invention generally relates to medical devices for use within a cavity of the human body, more particularly, indwelling drainage devices, and still more particularly, to urinary stents that dwell in the male prostatic urethra.

### BACKGROUND OF THE INVENTION

Discharge of bladder contents can be a source of serious and distressing problems for persons whose anatomy is temporarily, or over time, incapable of completely controlling the outflow of urine from the bladder, a clinical condition known as urinary retention. Traditionally, indwelling urethral catheters (i.e., Foley catheters, or the like), in which a free passage is created between the bladder and the outside of the human body so as to ensure the permanent flow of urine, have long been used to facilitate bladder drainage in individuals who are unable to initiate, or control such drainage, due to organic disability, immobility, or other physical impairment, most typically scenarios of acute, rather than chronic, retention.

For instance, acute retention is frequently experienced by patients who have recently undergone surgical intervention, either unrelated or related to the urethra. Acute urinary retention is also frequently experienced following radioactive seed implantation within the prostate, cryogenic treatment of the prostate, or minimally invasive procedures performed for the purpose of reducing the volume of the prostate. These include various thermal procedures such as the introduction of microwave energy, heat introduction systems, and chemical injections.

Urinary problems can have serious consequences, particularly when the problem is one of retention, incomplete emptying, or dysuria. Retention can result from any of a number of causes, including without limitation, spinal cord injury, typhoid, peritonitis, prostatic enlargement, urethral stricture, urethritis, cystitis, bladder tumors, or urethral calculus. Patients suffering from these, and other conditions, often require some interventional means to periodically drain or augment drainage of the bladder. Failure to do so can result in damage of the epithelium and detrusor muscles associated with the bladder, and an increased potential for bacterial invasion which is commonly thought to contribute to urinary tract infection (UTI) potentially leading to life-threatening kidney failure.

As individuals age, particularly males, the frequency of difficulties experienced within the intricate urogenital system increases. Problems range in severity from minor inconvenience, which lowers the quality of life, to life threatening disease. The following categories of medical conditions are noted: (1) bladder, or more generally, lower urinary tract dysfunction; (2) infection of the urinary tract; (3) infection and disease of the reproductive organs and glands; and, (4) life threatening disease of the urogenital tract.

Normal urine emptying for the human male or female occurs when the circulatory system, brain, spinal cord, kidney, ureters, bladder, prostate, urethra, and external sphincter are all healthy, and cooperate. The creation of urine occurs in the kidneys. The kidneys are positioned near the level of the first lumbar vertebrae. Stated in brief, the role of the kidney is to remove from the blood, water solutes and electrolytes, balance their concentrations, and create extra cellular fluid which is referred to as urine. The urine is comprised of nitrogenous waste, toxins (e.g. bacteria), water, and mineral salts. The urine passes through the ureters to the bladder.

The bladder serves two primary functions. First, it serves as a reservoir for urine. Second, it provides the pressure necessary to discharge the urine through the urethra.

The bladder is a compliant container or reservoir which resides behind the symphysis pubis bone. While the bladder is a thin walled structure, it is a complex structure which is comprised of three layers of smooth muscle, together known as the detrusor muscle, which form its walls, and contains mucous membranes which form a lining thereof. The bladder muscle is capable of a large measure of elasticity as the bladder fills. The ureters enter the bladder at the rear corners of the triangle-shaped floor (i.e., trigone) and the urethral opening at the front lower corner. This trigone region also has a higher concentration of nerves than the rest of the interior of the bladder.

Voluntary emptying of the bladder, referred to as micturition, normally occurs when an individual is aware that the bladder contains sufficient levels of urine. This mental awareness occurs because the urine stimulates stretch receptors in the bladder wall. This begins reflex contraction of the bladder wall, subsequent relaxation of the internal sphincter, and rapid relaxation of the external sphincter. The urine will then pass from the bladder to the urethra, and exit the body. This sequence of events can be withheld voluntarily, when, by the will of the individual, the neurological link is normal. Voluntary control is a learned response which depends on contraction of the external sphincter. Control relies on the nerves supplying the bladder and urethra, the projection pathway through the spinal cord, and the brain, the motor area of the cerebrum all being normal.

When an individual is unable to empty their bladder, the condition is called urinary retention. The causes for retention are either excess outlet obstruction within the urethra, or inability of the micturition process to progress in a normal coordinated manner.

The condition of excessive outlet obstruction is attributed to either benign prostate hyperplasia (BPH), more simply, enlarge prostate, or to malignant (i.e., cancerous) tumors. These conditions both produce enlargement of the donut-shaped prostate gland about the urethra. Generally, BPH develops with age when the prostate increases in thickness and length. This change places pressure on the urethra which it encompasses. The clamped urethra partially or completely restricts the flow of urine from the bladder. Cancerous tumors, which are tangentially discussed throughout, are discussed in greater length in co-pending application Ser. No. ______ entitled MEDICAMENT ARTICLE, ACCESSORY & SYSTEM filed Jun. 20, 2006, incorporated herein by references in its entirety.

Individuals who cannot easily and completely empty their bladders almost always experience a reduction in their quality of life. Patients are uncomfortable, fatigued, inconvenienced, insecure and discouraged, typically experiencing two or more of the following symptoms: (1) incomplete bladder emptying following urination which may result in abdominal discomfort; (2) constant feeling of needing to repeat urination (i.e., the “frequency” problem); (3) needing to strain to begin urination; (4) a burning sensation during urination; and, (5) fatigue caused from sleep deprivation associated with incomplete bladder emptying. Individuals with BPH may get up three or more times a night, urinating only in a small amount each time. Social behavior is often altered since the individual constantly feels a need to be close to a lavatory.

Up to two million office visits annually in the United States are attributed to patients being bothered by some form of lower urinary tract symptoms (LUTS). There are two primary organs, and the prostate, involved with the event of urination. The symptoms are virtually always suspected to be caused by the intrusion of an enlarged prostate gland upon the urethra, however, symptoms are often caused by irregularities in bladder function, or sphincter deficiencies. For this reason, bladder outlet obstructions (BOO) is a major subgroup of LUTS. In men between the ages of 55 and 75 years, it is estimated that between 50 and 75% have some degree of bladder outlet obstruction, however, it may not be responsible for their symptoms.

As previously noted, bladder outlet obstructions are primarily caused by the enlargement of the prostate gland which results in radial compression of the urethra surrounded thereby (i.e., the prostatic urethra), thus obstructing (i.e., constricting) urine flow, resulting in incomplete emptying of the bladder (i.e., there being what is clinically referred to as a “post void residual” (PVR) remaining in the bladder). Heretofore, males presenting with LUTS have few diagnostic options prior to either long term pharmacological, or invasive irreversible medical procedures such as trans urethral resection of the prostate (TURP), or non-surgical procedures such as thermal treatment of the prostate.

Physiological endourethral device consideration are numerous. For example, and without limitation, prostatic urethra lengths vary greatly from person to person; the urethra can contract and extend slightly in length throughout the course of a day (i.e., the urethral environment is dynamic, or alternately, suffice it to say it is not a static or constant environment); the bladder base is sensitive to contact due to among other things, the presence of the trigone nerve region; both the bladder and urethra contract in response to physiological requisites; and, urinary salts and bacteria are present within the lower urinary tract.

The urinary, neural and reproductive systems are interactive or interlinked. Diseases, injuries or malfunction within the bladder, prostate or spinal cord will affect normal removal of urine from the bladder. Furthermore, diseases within the prostate may also affect function of the reproductive system by obstructing the ejaculatory ducts that pass through the interior of the prostate. The bladder contraction and internal sphincter relaxation are controlled by the nervous system. When the spinal cord or nerve network is damaged, or incompetent, normal emptying is interrupted.

The male reproductive fluid tract is interconnected with the urethra. Semen, ejaculatory fluids and glandular secretions also leave the body through the urethra. These fluids enter the urethra within the prosthetic portion, and downstream thereof. The orientation of the prostate, which is located anterior of the rectum, and seminal vesicles, can result in physiological interdependencies which become complex to diagnosis and treat.

Physiological balances or tensions in the urinary system are believed less stable as an individual ages. This is particularly true with casual diuretics such as coffee may cause urgency and frequency. Another common imbalance, constipation, places pressure on the posterior of the prostate adding to the effect of obstruction, with many drugs known to have a side effect of constipation.

In benign disease, the cause of symptoms is not easily identified. A leading abnormality is prostatitis. In a first form of prostatitis, a bacterial infection is associated with inflammation at the outlet or base of the bladder and prostate, with the administration of a common antibiotic being a standard treatment approach. A nonspecific form of prostatitis is also known, having similar symptoms to the bacterial form. Symptoms of urgency, frequency, and difficulty urinating are generally treated via local administration of solutions to quiet and sooth the bladder.

To illustrate the cooperation of the prostate gland with the reproductive system, men with severe BPH are known to experience a reduction in seminal ejaculate, or the ejaculate may be routed to the bladder. This is clinically referred to as retrograde ejaculation. This abnormality occurs as a result of the prostate either impinging upon the seminal vessels which route into the prosthetic urethra, or by the obstruction of the urethra resulting in the path of least resistance for the seminal discharge, namely, the upper urethra which is the bladder outlet.

The most serious of all diseases affecting the lower urinary tract is the presence of cancer, for example, prostate cancer. Prostate cancer is generally viewed as a disease that presents years or decades subsequent to earlier benign inflammations and/or symptoms. Scientific articles speculate that more then half of men over fifty years of age have some cancer cells in their prostate. Like most cancers, the treatment response depends upon “the state”. Early stage prostate cancer is defined as the cells being entirely encapsulated within the prostate “capsule”. Prostate cancer is an unusual cancer because it can remain in the prostate capsule for as long as twenty-thirty years. When this occurs, the patient may not know that it is present, or have any symptoms. The capsule may however, enlarge and produce similar voiding symptoms to those of benign enlargement.

Because prostate cancer is a disease that often occurs late in life, the patient may have prostate cancer but die of an unrelated affliction. For this reason prostate cancer is difficult for the urologist or oncologist to known when to treat.

When an intervention is undertaken, the intervention is normally to either surgically remove the prostate in a procedure called a radical prostatectomy, or try to kill the cancer cells in place. The latter is done by either injecting radioactive seeds into the prostate to attack the cancer cells, or try to locally freeze the cancer cells with a procedure called “cryo-therapy,” essentially a local freeze drying of the prostate tissue.

When the prostate cancer advances to a stage where the tumor has grown through the prostate capsule, it is considered late stage disease. At this stage, the cancer has great potential to be metastatic. In other words, it may attached and grow into and through adjacent bones, organs, or muscles. Generally, chemotherapy or external beam radiation will be used in effort to stop the progression of the cancer to surrounding physiological structures.

Devices have been developed to be positioned in the urethra and/or bladder to correct the problems of urine flow. Problems and disadvantages of heretofore known devices include the deleterious effects (i.e., pitting, depositions, etc.) associated with the urethral environment upon critical device components (e.g., valve actuators, flow conduits, etc.) which at a minimum render such devices less effective, and which at a maximum, cause device component failure, or render the device wholly ineffective, which necessitates emergent removal and, as the case may' be, urinary tract damage repair. Problems of device leakage, or less than complete emptying of the bladder are also widely known. Furthermore, issues surrounding device deployment and fit, positioning, repositioning, and retention (i.e., sufficient anchoring) have also been well documented.

It is especially critical that the endourethral device be stable with respect to position (i.e., a physiologically properly deployed and stable position), and comfortable to wear, as the urinary tract is sensitive to contact. Inter-urethral stents have been utilized within the male urethra within the prostatic region with many users foregoing such devices for alternate therapies due to feelings of discomfort and/or pain. Many endourethral devices have similarly been evaluated for urinary incontinence for females. Based upon clinical findings, many have been shown to be uncomfortable, thus severely retarding their utility as a therapy. Other devices have migrated into the bladder, or have been expelled under straining conditions.

Furthermore, it is imperative that the device be no more invasive as is necessary. For instance, it is advantageous that the device minimally engage the structures of the lower urinary tract, particularly in accomplishing an anchoring function. For example, it is well known that secretions of the prostatic urethra, including the Cowper's glands, whether during sexual function or otherwise, is clinically beneficial. The secretions are comprised, in part, of antimicrobial agents which assist in the prevention of urinary tract infections. It is further believed that bathing of the bladder neck with urine assists infection prevention. Generally, flow of urine external of an endourethral device permits the free passage of urinary tract fluids from the urethra as urine is released, thereby allowing a more physiologically normal urine discharge. Thus, whether it be a short or long term endourethral device, for interventional, diagnostic or other purpose, stable anchoring in combination with physiologically proper, non-traumatic device deployment and retention is essential.

### SUMMARY OF THE INVENTION

A self adjusting and/or positionable indwelling urethral device is provided. The device generally includes a prostatic urethral stent body, and a urethral anchoring element. The prostatic urethral stent body includes a preconfigured end portion for anchored receipt within a bladder, with the urethral anchoring element extending from the prostatic urethral stent body, as for example via a linkage, or more generally, an operative connector.

The preconfigured end portion of the prostatic urethral stent body is advantageously preformed or pretensioned. The end portion is generally delimited by a point of transition in the stent body, more particularly, a bend or curvature.

In first and second embodiments of the subject invention, the preconfigured end portion is formed as a “rolled-up” free proximal end. The rolled-up or spiraled free proximal end may be axially aligned with an axis of elongation of the stent body, generally appearing as a “tee” in elevation view, or contrariwise, the spiraled free end may be axially orthogonal in relation to the axis of elongation of the stent body, i.e., the stent body generally appears as a linear “tail” segment of the spiral. It is to be noted that proximal anchor structures delimited by the subject preconfigured end portion may have an angular orientation between those described. In a further embodiment, the preconfigured end portion is formed so as to generally resemble a candy-cane, i.e., the free end segment includes a curved terminal end spaced apart from the point of transition via a linear body segment.

The novel devices described hereafter support the male urethra in an open condition, and permit unencumbered urination. The subject devices also offer relief from the discomforts of obstruction and diagnostic utility. Several of the embodiments are selectively useful for relief of symptoms associated with overactive bladder, prostatitis, and treatment of infection and cancer within the urogenital tract.

Further still, the subject devices offer many clinical advances. First, all embodiments provide improved urine drainage in patients with obstruction by supporting the prostatic urethra in an “open” status regardless of the reason for the obstruction. Second, all embodiments provide ease of insertion and self adjustment of the device. Third, all embodiments provide for convertible features allowing for continuous drainage or normal physiological drainage. Fourth, all embodiments provide two point anchoring. Fifth, all embodiments provide for an ideal platform for medicament, e.g., solution, delivery. Sixth, use of the subject devices provide for treatment and diagnostic opportunities that do not presently exist.

The subject devices are to be placed within the human urethra in communication with the bladder. Although the subject disclosure details specifics of the male anatomy, it is nonetheless obvious to those skilled in the art that several embodiments directly, or with minor derivation, offer advantage to the human female. Females also frequently suffer from urinary tract infections and cancer, and even occasional urinary retention even in the absence of a prostate gland.

Each device embodiment may be easily positioned to accommodate the prostate length and sphincteric anatomy of the patient. Devices are stabilized in the urethra by two anchoring elements. The first anchor is preferably positioned in the bladder; a second anchor is positioned within the bulbous urethra. The anchor portions are spaced apart by a body which selectively supports a portion of the urethra without inhibiting the normal function of the external sphincter. The devices therefore re-enable physiologically normal urination. The device configurations are easy to insert, stable, and easy to remove. In connection to removal, a preformed or pretensioned free end of the device may be readily manipulated so as to “release” the configuration associated therewith in furtherance of device removal ease and/or patient comfort.

Placement without external visualization is accomplished with the unique system which is comprised of the insertion device and the device together. There is no need for either a cystoscope to be inserted into the urethra through the penis, or for a transrectal ultrasound procedure to be performed to confirm proper orientation.

The devices of all embodiments may be installed in similar fashion to a Foley catheter, namely, by simply: inserting the device until the free end or tip is well within the bladder; withdrawing the device into the bladder outlet; and, removing the insertion device.

Additional items, advantages and features of the various aspects of the present invention will become apparent from the description of its preferred embodiments, which description should be taken in conjunction with the accompanying drawings.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles, elements and interrelationships therebetween of the invention.

FIG. 1 is a cross-sectional depiction of male anatomy generally illustrating, among other things, the physiological structures of the lower urinary tract;

FIG. 2 generally illustrates select physiological structures of FIG. 1 as viewed from the “front”;

FIG. 3 provides a perspective view of an endourethral device of the subject invention;

FIG. 4 illustrates a variant of the device of FIG. 3 in a deployed or indwelling condition within the lower urinary tract;

FIG. 5 illustrates a perspective view of an alternate embodiment of the endourethral device of the subject invention;

FIG. 6 depicts a variant of the device of FIG. 5 in a deployed or indwelling condition within the lower urinary tract;

FIG. 7 depicts a further embodiment of the endourethral device of the subject invention;

FIG. 8 illustrates a variant of the device of FIG. 7 in a deployed or indwelling condition within the lower urinary tract;

FIG. 9 illustrates a catheter system of the subject invention, more particularly, a device of the subject invention in combination with an insertion tool;

FIG. 10 illustrates the assembly of FIG. 9 during device placement procedures, i.e., relative to structures of the lower urinary tract;

FIGS. 11-14 generally illustrate the device of FIG. 3 equipped with means for delivering a medicament to at least a portion of the lower urinary tract; and,

FIG. 15 depicts the device of FIG. 7 equipped with alternate means for delivering a medicament to a portion of the lower urinary tract.

### DETAILED DESCRIPTION OF THE INVENTION

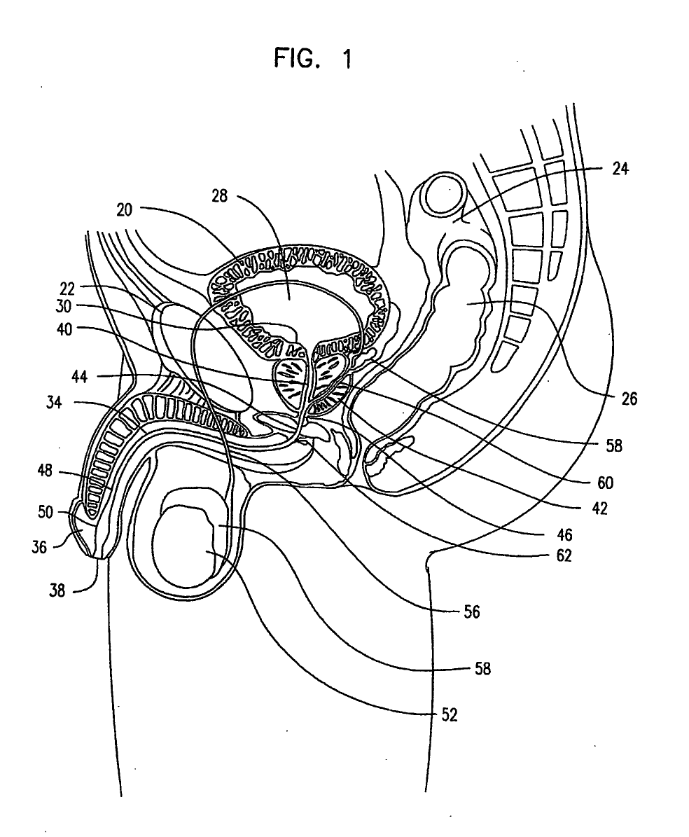

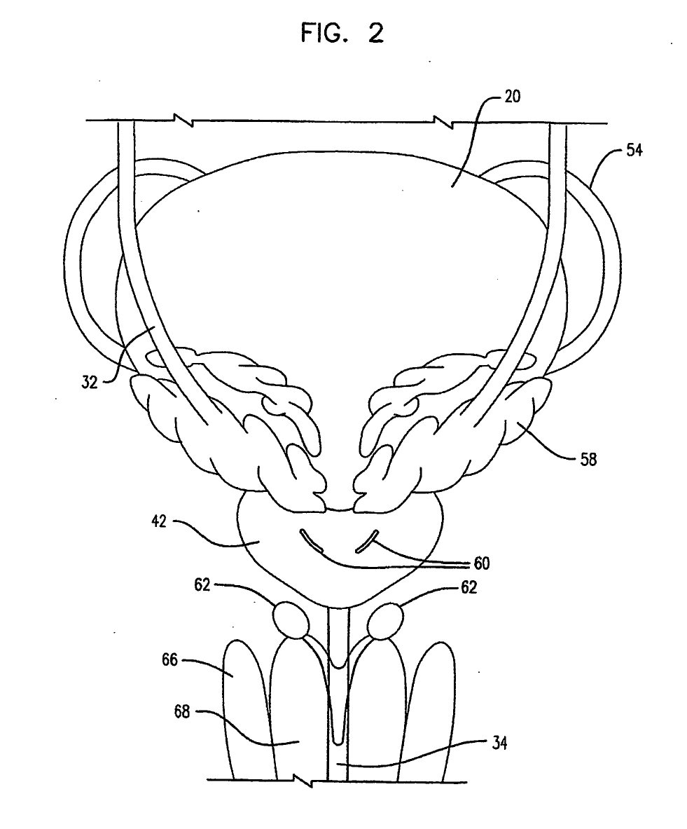

Prior to a detailed discussion of the subject device, its several embodiments, and attendant systems, an abbreviated description of the anatomical environment of same is helpful. In furtherance thereof, FIGS. 1 & 2 generally illustrate the physiologic structures of the male urinary system, as well as the male reproductive system. A sectional side view of the male urinary system is presented in FIG. 1 with a front view of select structures of FIG. 1 illustrated in FIG. 2.

In connection to the urinary system, the bladder 20, generally centrally located and residing posterior of the pubic bone 22 and anterior of the sigmoid colon 24 and rectum 26 (FIG. 1), temporarily stores urine 28, and periodically expels it when the bladder neck 30 (i.e., the lower base of the bladder) opens, as the bladder 20 contracts. Urine, produced by the kidneys (not shown), passes into the bladder via dedicated ureters 32 (FIG. 2), and periodically exists therefrom via the urethra 34, a continuous passageway which extends from the bladder 20 to and through the penis glands 36, terminating at the urethral opening 38.

Urine passes through the prostatic urethra 40, which is completely surrounded by the prostate 42. The distal limit of prostate 42 is marked by a small projection called the verumontanum 44. This is an important landmark because distal thereto, is the external urethral sphincter 46, which relaxes prior to the urination process beginning. Beyond this is the penile urethra 48, affording a free passage of urine external to the body, beyond the external urethral meatus 50.

It should be appreciated that the subject anatomical systems are “dynamic,” for example, although the bladder neck appears fixed and funnel shaped, the reality is that this structure is highly mobile. The bladder neck twists and turns as the bladder fills and empties. Under normal conditions, the bladder neck twists as it closes. In part, the function of the bladder neck is to cooperate with the external sphincter to restrict urine from involuntary drainage. Finally, there is a network of nerve endings (not shown) at the base of the bladder, the trigone, which are very sensitive to contact, and which are almost continually active.

The trigone, which is a smooth sensitive triangular region of the internal urinary bladder, is formed by the two ureteral orifices, and the internal urethral orifice. The area is very sensitive to expansion, and once stretched to a certain degree, the urinary bladder signals the brain of its need to empty. The signals become stronger as the bladder continues to fill. Clinically, the trigone is important because infections tend to persist in this region. Furthermore, the trigone is known as an inherently sensitive area.

In connection to the reproductive system, testes 52 provide reproductive sperm to the vas deferens 54 via the epididymis 56. The stored semen of the epididymis is subsequently mixed with diluting fluid from the seminal vesicle 58, and other accessory glands, subsequent to entering the ejaculatory duct 60, so as to form semen prior to ejaculation. The ejaculatory ducts are formed by the union of the seminal vesicles 58, which are short tubes descending through the prostate gland 42 and into the urethra 34, and the vas deferens 54. The bulbourethral glands 62 (i.e., Cowper's glands), located behind and lateral to the membranous portion of the urethra, secrete a clear fluid known as pre-ejaculate or Cowper's fluid which is generated upon sexual arousal. The excretory duct of each of these glands are approximately 2.5 cm long, passing generally forward, beneath the mucous membrane on the floor of the cavernous portion of the urethra, about 2.5 cm in front of the urogenital diaphragm.

Finally, in connection to the penis 64, the corpus cavernosum 66 and corpus spongiosum 68 are three expandable erectile tissues along the length of the penis 64 which fill with blood during erection. The two corpora cavernosa 66 lie along the penis shaft, from the pubic bones 22 to the head of the penis, where they join. These formations are made of a sponge-like tissue containing irregular blood-filled spaces lined by endothelium and separated by connective tissue septa. The corpus spongiosum 68 is one smaller region along the bottom of the penis 64, which contains the urethra 34 and forms the glands penis 36.

As will be subsequently appreciated in connection with the detailed description of the subject invention, to a great extent, the importance or significance of same may be credited to the criticality of the anatomical region it serves. Figuratively, the previously described region of the urethra is comparable to a busy street with critical intersections. The reproductive system converges with the urinary system. Remarkably, the rectum 26, which lays posterior of the prostate 42, is in close enough proximity thereto that it too affects the emptying or discharge of urine, and even ejaculation. Essentially there exists four chemical/biochemical highways: the urethra; the seminal vesicle; the ejaculatory duct; and, the Cowper's glands. All the subject structures, and their related passageways, are in or near urine as it descends from the bladder. Disease and infection often affects the entirety of the systems.

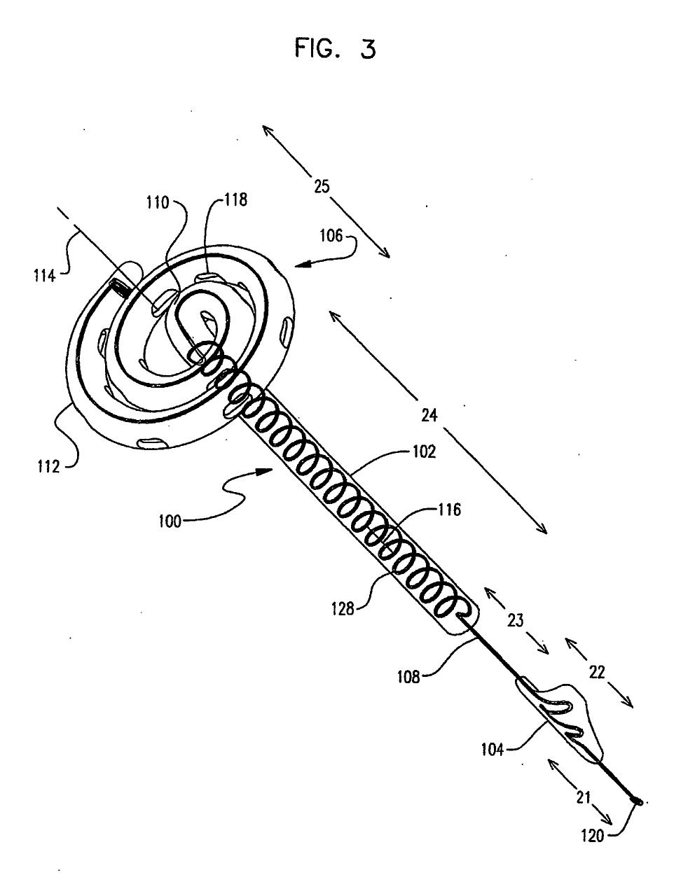

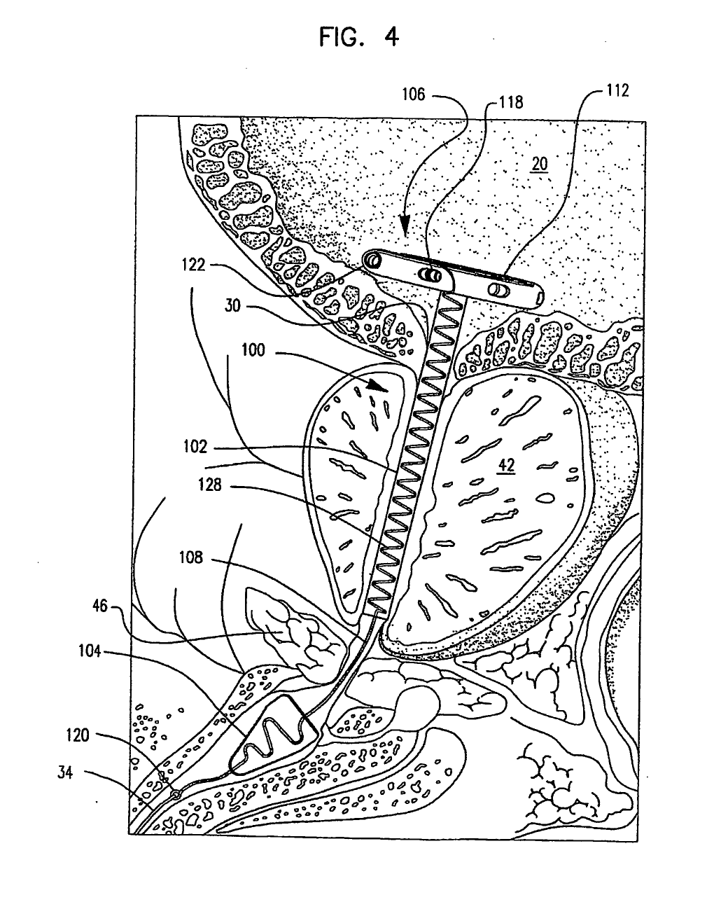

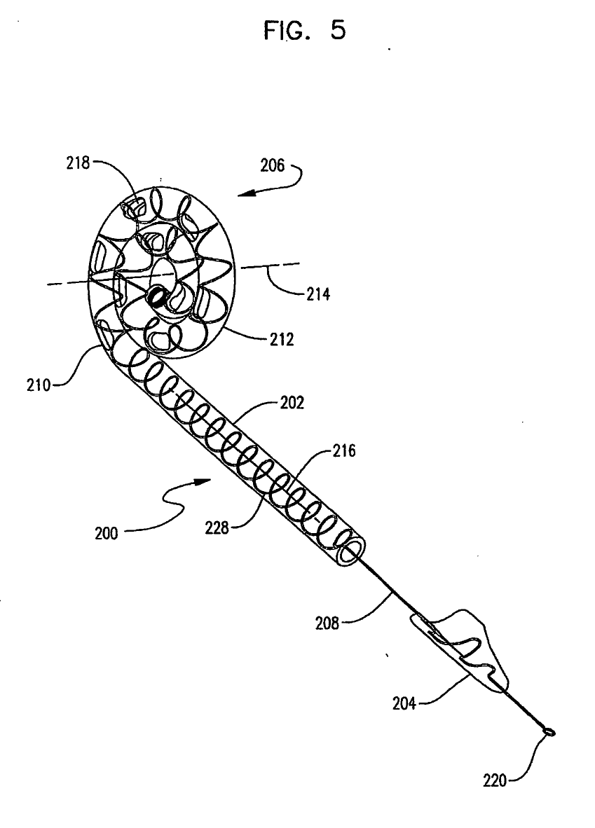

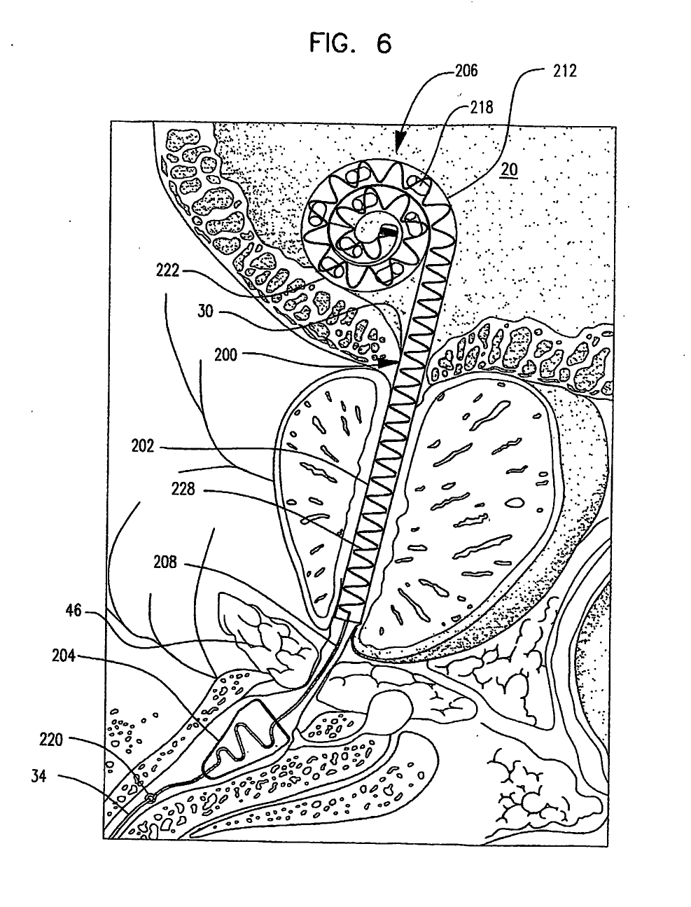

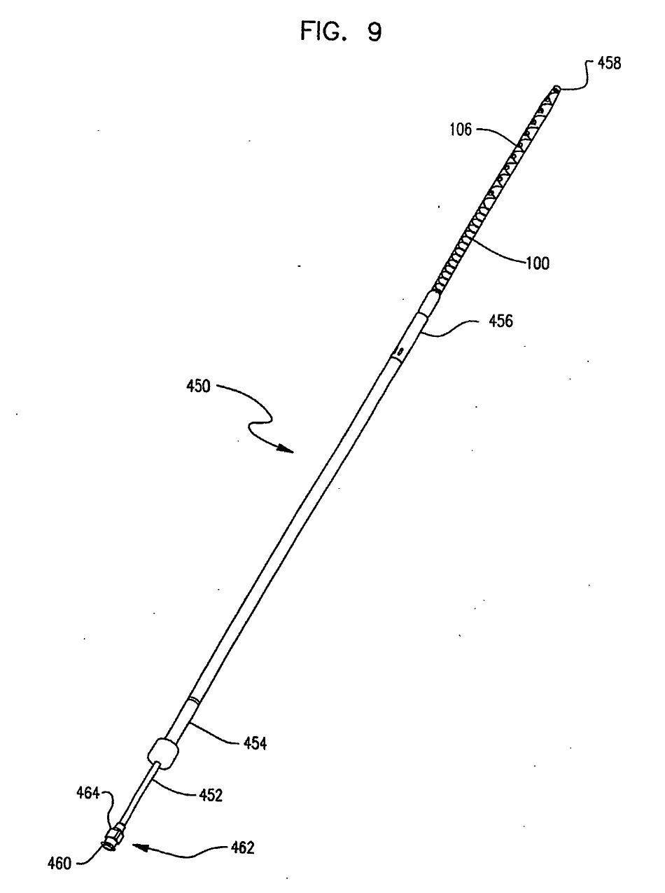

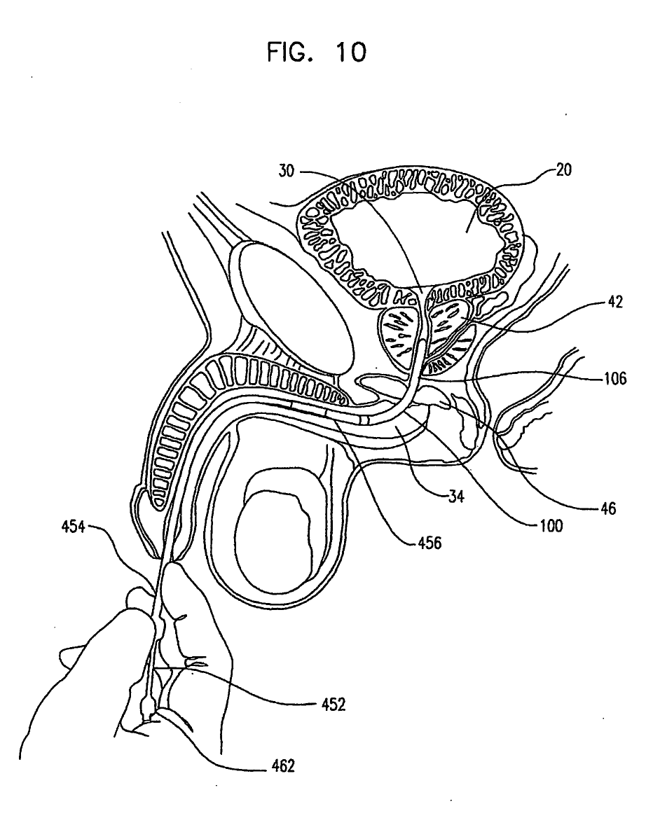

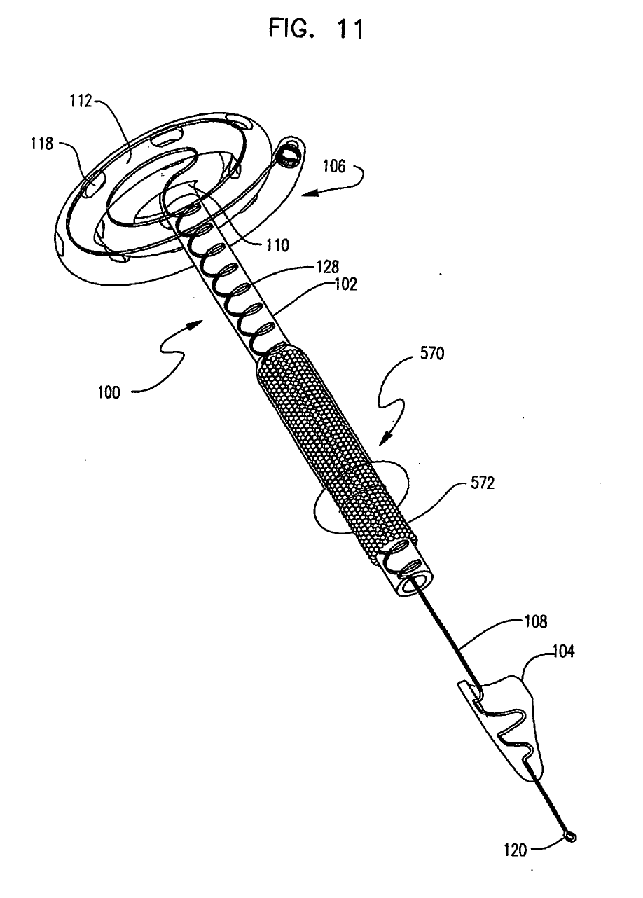

Illustrative, non-limiting embodiments of the subject invention per se are shown in FIGS. 3, 5, & 7, with companion figures, namely, FIGS. 4, 6, & 8, generally illustrating variants of said embodiments in situ, i.e., in a fully deployed or indwelling condition within the urinary tract. A catheter system, generally comprising an indwelling device and insertion tool, is illustrated in FIGS. 9 & 10. As will later be explained, the system, in addition to a device delivery functionality, has utility as a bladder drainage device. Finally, the non-limiting embodiments of FIGS. 3, 5, & 7 are illustrated in FIGS. 11-15 equipped with a variety of medicament delivery means, i.e., medicament carrying structures (see co-pending application Ser. No. ______ entitled MEDICAMENT ARTICLE, ACCESSORY & SYSTEM filed Jun. 20, 2006, for further details).

With general reference to FIGS. 3, 5, & 7, and particularly FIG. 3, there is generally shown an indwelling urethral device 100 (i.e., device 100, 200, or 300 respectively, having reference numerals +100, +200 or +300 respectively for like structures) characterized by a prostatic urethral stent body 102 and a urethral anchoring element 104. The urethral stent body 102 includes a preconfigured or pretensioned end portion 106 for anchored receipt within a bladder, with the urethral anchoring element 104 extending from the prostatic urethral stent body 102 via a linkage, e.g., structure or element 108, for indwelling placement within the bulbous urethra.

Notionally, the subject device, in all its embodiments, can be viewed as having five (5) zones, the zones generally corresponding to anatomical locations within which portions or segments of the device indwell, namely: (1) penile urethra; (2) bulbar or bulbous urethra; (3) external sphincter; (4) prostatic urethra; and, (5) bladder, more particularly, bladder neck. For the most part, the following device structures/zonal relationships are noted: retrieval means (zone 1); distal anchor (zone 2); linkage, i.e., tensile member (zone 3); stent body (zone 4); and, proximal anchor (zone 5).

As should be readily appreciated, the directional terms proximal and distal require a point of reference. Throughout the subject description, the point of reference in determining “direction” is in the perspective of the patient. Therefore, the term proximal will always refer to a direction that points into the patient's body, whereas distal will always refer to a direction that points out of the patient's body.

As previously noted, the prostatic urethral stent body 102 generally includes a pretensioned or specifically configured resilient end portion 106 for anchored receipt within a bladder, and a distal anchor 104 extending therefrom, as by a link, linkage, tether, etc. It is to be noted that functional and structural particulars for elements distal of the stent body (i.e., those associated with zones 1-3) are detailed in one or more the following published U.S. patent applications and/or U.S. patents, namely, Pub. No. US 2002/0107540 A1 and U.S. Pat. No. 6,991,596 B2, each of which is incorporated herein in their entireties. The focus of the remainder of the subject description will be primarily directed to zone 4 & 5 device elements.

While the purpose of anchoring the subject and similar devices is covered in Applicant's cited application(s) and/or patent(s) more fully, it is again to be emphasized that the bladder outlet is dynamic and very active during the urination process. Activity of the bladder outlet may move an endourethral device proximally toward the bladder. Conversely, an indwelling endourethral device may be moved distally due to the force of the urine being discharged through and about the device. Devices of all embodiments of the subject invention are advantageously, but not necessarily, provided with anchoring structures to prohibit distal and/or proximal device displacement.

In connection with proximal anchoring of the devices of the subject invention, three primary functionalities are noted for same: (1) facilitation of urine egress from the bladder; (2) provisions for self-adjustment for stent body placement (i.e., a retraction or extension fit for the device); and, (3) provisions for a bladder neck “friendly” structure, i.e., a structure configured to eliminate/minimize trigone area irritation.

With particular reference now to FIGS. 3 & 4, first embodiments of the subject device are illustrated. The pretensioned, non-linear end portion of the prostatic urethral stent body 102 is delimited by a bend 110. The pretensioned end portion 106 generally comprises a rolled-up free end for the prostatic urethral stent body, more particularly, the prostatic urethral stent body is characterized by a spiral end segment 112, i.e., a segment or portion which turns around a central point or axis 114, getting progressively closer to or farther from it, depending upon which way one “follows” the segment. Although generally illustrated as a two-dimensional spiral, the configuration need not be so limited, i.e., the end portion may be configured as a three dimensional spiral, or variant thereof, e.g., a coil, helix, conic structure, etc.

The subject configuration for the end segment 106 of the prostatic urethral stent 102 generally delimits a substantially planar or disk-like bladder anchor which inherently, via its configuration and/or materials of construction, imparts a resiliency for the stent body 102 so as to be responsive to physiological activity of the environmental anatomy.

As shown, the anchor generally has a lateral extent substantially perpendicular to an axis of elongation 116 of the stent body 102. Again, it is to be readily appreciated and understood that the configuration of FIG. 3 is inherently associated with that of the “static” device, however, a deployed configuration for the pretensioned end portion 106 of the stent body 102 may appear as a three dimensional or non planar element as opposed to the two dimensional spiral illustrated in FIGS. 3 & 4.

In connection to the pretensioned or rolling end portion 106, a series of ports 118 are located therethrough. The rolling section provides passage of the urine from the bladder to and through the stent body; provides self adjustment rolling or coiling to take up extra length; and, it is generally configured to spread out anchoring forces throughout and away from the trigone nerves. In connection with FIG. 4, the stent body 102 extends from the rolling section 106 through most of the prostate 42, with the tensile member 108, which may be reversible extendible, resilient, or possess memory properties, extending therefrom and through the external sphincter 46 through the distal anchor 104. In-as-much as distal anchor related details are beyond the immediate scope of the present description, and elsewhere provided as noted, the tensile member 108 may be a single strand or filament (FIG. 3) or a combination or combinations of a filament or a suture (FIG. 4) which extend through the distal anchor 104 and terminate in retrieval means 120 for the device, or which alternately terminate at or within the anchor per se.

The pretensioned end or rolling section 106 prohibits movement of the device 100 distally from the bladder 20 via contact with the bladder outlet 30, and to a lesser degree the prostate 42. Likewise, the device is prohibited from proximal movement or displacement into the bladder by the distal anchor 104. The tensile linkage 108 extending between the distal anchor 104 and the stent body 102 spans the gap across the external sphincter 46 (FIG. 4), with such configuration and/or arrangement being readily adapted or adaptable for adjustment in furtherance of improved patient fit/comfort. The nature of the linkage 108 is such that it is sufficiently compliant and small in cross section that it allows for complete closure of the urethra 34 beneath the external sphincter 46: urine only leaves the bladder by the patients initiative, complete control is maintained by the patient.

With particular reference now to FIGS. 5 & 6, second embodiments 200 of the subject device are illustrated. For the most part, the present device embodiment generally mimics that of FIG. 3, a point of departure being the orientation of the pretensioned end portion 206 of the prostatic stent body 202.

As is readily appreciated with reference to the figures, the pretensioned end portion 206 “rolls up,” reversibly, along the axis of elongation 216 of the stent body 202, i.e., the anchor so configured is substantially co-planar with the stent body. This orientation offers an opportunity to place a point of bladder engagement or contact 222 near the periphery or outer portion of the trigone nerve region. Similar to the previously described device, the coiling (i.e., potential energy) of the free end portion 206 of the stent body 202, namely, the proximal anchor, provides a slight proximal motivation towards the bladder so that upon insertion, the device will align itself for positioning purposes.

In connection with the heretofore described devices, in-as-much as each might be characterized as having a static proximal anchor configuration appearing, arguable, as a spiral lollipop of several “turns” (with awkward “stick” placement, i.e., stick center and perpendicular (FIG. 3), and stick extending from the free-end of the spiral), the extent of spiraling shown is illustrative (i.e., should not be considered limiting). For example, the proximal anchor may include only a single turn, curve or bend.

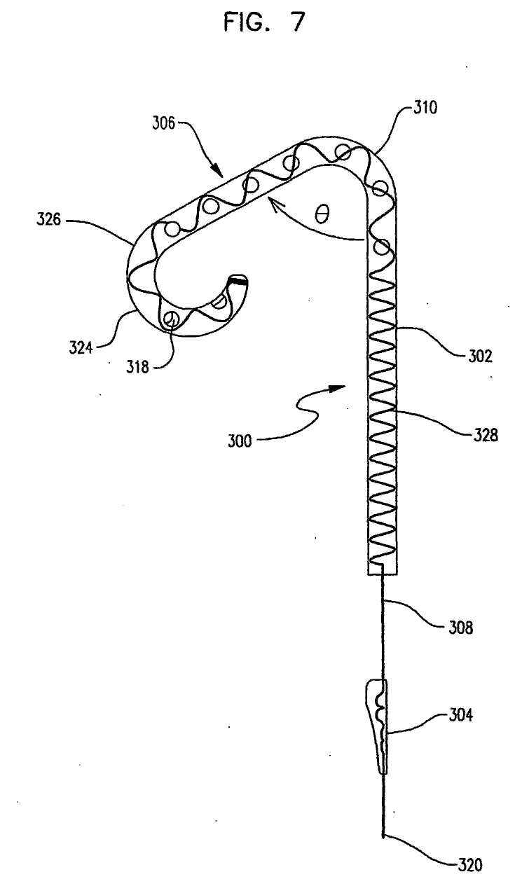

With particular reference now to FIGS. 7 & 8, third embodiments 300 of the subject device are illustrated. As the case with previously discussed embodiments, the pretensioned or loaded/pre-loaded free end 306 of the stent body 302 is adjacent, i.e., proximal, a bend or sweep 310, more generally, a transition point. As illustrated, the “static” bend is at an angle θ of about 60°, with the angle θ being within a practical range of about 30-135°, a preferred range being within the range of about 45-90°. In a deployed condition (i.e., a “dynamic” device status), the angle θ is variable due to the physiological responsiveness, and associated activity, of the elastic transition point 310, or the device 300 more generally. The responsiveness of the transition point provides the sought after device positioning motivation.

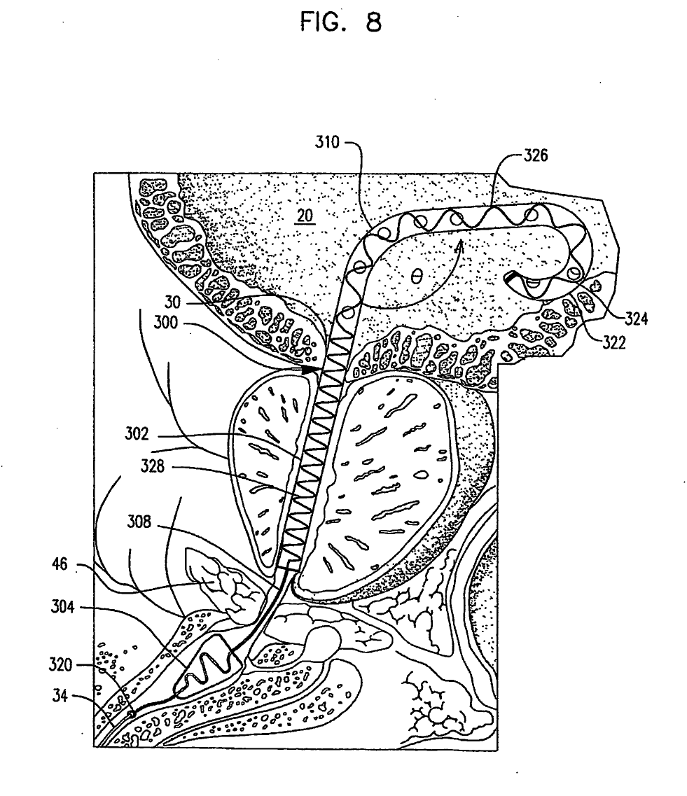

Characteristic of the proximal anchor of the subject embodiment is a hooked (i.e., curved) free end segment, more particularly, a hooked free end segment 324 which is spaced apart from the transition point 310. Intermediate the transition point 310 and the hooked or hook-like free end segment 324 is a linear segment 326, advantageously, but not necessarily, within the range of about 2-5 centimeters (cm) in length. As should be readily appreciated with reference to FIG. 8, such arrangement in a proximal anchor defines a point of bladder engagement or contact 322 for the anchor outside, or at least remotely, as may be practical, from the trigone region.

In connection to the heretofore described devices, fabrication or construction materials preferably, but not necessarily, include medical grades of silicones or polyurethane, known family members thereof, and other suitable alternatives as the case may be. Silicones are considered safe, and easily accommodate reinforcement structures to the extent they are contemplated. Similarly, polyurethanes are advantageous, whether in connection as a device “base” or in connection to medicament delivery functionality. It is contemplated that such reinforcement structure or structures (e.g. 128, 228, 328) be advantageously, but not necessarily constructed of 304V stainless steel, or nickel-titanium (Ni/Ti) alloys generally referred to as Nitinol, polymeric or fiber composite materials. The use of the nickel-titanium alloys in the devices of the subject invention provide added “memory.” Finally, it is to be appreciated, in the context of the subject invention, and endourethral device more generally, that reinforcement structures or elements are generally predicated upon the notion that “form fits function,” e.g., a non-constant coil pitch or other formations providing engineered deformation, e.g., serpentine springs or the like, may be advantageous utilized in the stent body due to utilitarian departures in the prostatic segment of the stent body on the one hand, and the pretensioned free end thereof (i.e., the proximal anchor) on the other.

The outer dimensions of the device bodies of the described embodiments are within the range of about 10-20 cm in length, though they need not be so limited. The portion or segment which extends from the bladder outlet to the external sphincter, i.e., zones 2-5, will generally range from about 4-12 cm. The tensile member may range in length from 1.7-5 cm, with a preferred length of 2.0-3.0 cm. The outer diameter of the device body may range from about 12-30 French, with a preferred range of 18-22 French. Finally, while the wall of stent body is constructed to optimize the internal diameter, its thickness is preferably less than about 0.030 inches (in) for the described devices.

Referring now to FIGS. 9 & 10, a catheter assembly 450 or system is shown. The system generally includes an endourethral device as heretofore described e.g., device 100, positioned upon a free end of an insertion device or tool. The insertion device advantageously includes cooperatively engageable first and second concentric tubular elements or members, more particularly, an inner device support 452 translatable within an outer pusher 454 having a free end terminating in a sheath, more particularly, a anchor receiving structure 456 for essentially “housing” the distal anchor 104 of a “loaded” device 100.

The device support member 452, which, like the pusher 454 may include a reinforcing member or elements 428, may include an open device end 458, or is other otherwise adapted to permit two way communication, i.e., in/out, or guide wire interaction with a lumen 460 thereof. Opposite the device end 458 is an operator end 462, advantageously, but not necessarily characterized by a luer fitting 464 or the like.

As should be readily appreciated, the device support member 452 is of sufficient rigidity to retain the loaded device 100 in an elongate or extended configuration, i.e., a configuration wherein the pretensioned or preconfigured end portion 106 of the stent body 102 is “overcome”. As such, with a visual, tactile or other indicia of proper device placement, the device support 452 may be retracted relative to the pusher 454, thereby releasing the proximal end portion or tip of the stent body 102 which is predisposed to revert or return to the preselect configuration, thus enabling proximal anchoring of the device in the bladder.

In the course of device insertion, when the proximal extremity of the device reaches the bladder, urine will pass from the bladder, to and through the device, and thereafter, through the interior of the insertion tool via the lumen of the device support. This allows a visual confirmation that the device extremity. i.e., proximal tip, is sufficiently positioned in the bladder. The luer fitting advantageously provides regulated or controlled release of urine accumulating in the bladder until elective removal of the insertion device.

The central passageway or lumen of the inner member also allows for fluids to be directly introduced into the bladder prior to the removal of the tool. This is an extremely useful feature because of the complexities of diagnosing and treating bladder insufficiency and urinary tract obstruction.

For example, when the urologist or caregiver examines the patient, if they have had difficulty urinating, or are unable to entirely empty their bladder, the problem is usually attributed to an enlarged prostate impinging upon the urethra, and, it is presumed that the patient has sufficient bladder function. On the other hand, if patients present in acute urinary retention (AUR) they are able to drain little or no urine. Some of these patients will lack the ability to produce contraction within their bladder. When these patients receive a device of the subject invention, little or no drainage of urine will occur when the patient attempts to urinate.

As previously noted, the lumen system that includes the ports and inner member of the insertion device allows drainage of urine giving instant relief to patients in acute retention whose bladders still contract. This ability to differentiate by trial is very useful. This differing response is a very valuable function of the subject system or assembly.

Contrariwise, the continuous passageway within the interior of the insertion tool and the body may be used to fill the bladder prior to the removal of the insertion tool. This pre-filling enables the caregiver to get an immediate assessment of the competency of the bladder either before the insertion tool and device are uncoupled, or most effectively, immediately after the insertion tool is removed from the urethra. The measurement of the flow rate is a very simple and quick test which gives high confidence indicator of bladder competency. This “trial void” allows the physician to know that the patient is safe and comfortable prior to release.

The utility of the subject system or assembly is particularly advantageous to patients that present to an emergency room an acute retention, for post surgical patients, and for patients who have received a minimally invasive procedure to treat benign or malignant sores of the prostate. BPH and prostate cancer retention episodes may each require a period of recovery requiring passive urine drainage prior to the stenting phase. For example, following acute retention the bladder may be stunned and unable to contract as a result of over-stretching. When the patient has accumulated more than approximately 800 cc in the bladder, it is normal practice to allow the bladder to rest for a few days, or even up to a few weeks. The bladder is given this rest when the Foley catheter is placed in the bladder, allowing it to continuously drain. The subject system may advantageously be left fully integrated to provide the function of a bladder drainage catheter. Alternately, when clinically appropriate, the device may be separated from the insertion tool, and converted to provide a stenting function. The subject system provides the option of passive drainage or active drainage according to the perceived need, and likewise provides scheduling flexibility as the patient is referred to the urologist for follow-up.

Referring now generally to FIGS. 11-15, heretofore described devices of the subject invention are illustrated equipped with a variety of means for delivering medicaments or the like. As previously noted, a full description of the medicament deliver structures and their functionality are subject and focus of co-pending application Ser. No. ______ entitled MEDICAMENT ARTICLE, ACCESSORY & SYSTEM filed Jun. 20, 2006.

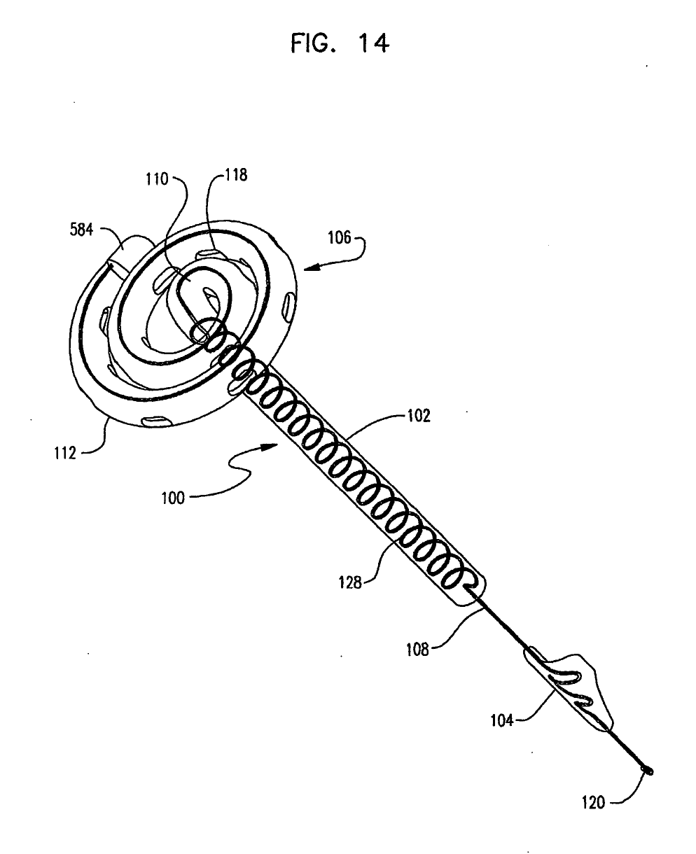

Notionally, the contemplated medicament deliver structures, i.e., adjunctive accessory articles or modules, are advantageously, but not necessarily (see e.g., FIG. 14) device conforming structures, such as sleeves or the like. As illustrated, the subject device conforming medicament deliver structures are intended to be carried by the stent body, more particularly, selectively carried thereby (i.e., the medicament deliver structures are preferably a discrete element, that is to say, not integral to the device, however, integration of the medicament delivery means into the device per se is likewise contemplated.

As is widely acknowledged, “drug” delivery systems require two essential functionalities, namely, (1) drug retention, and (2) predictable drug release/elution. In furtherance of such functionality, the subject system, i.e., laminate, preferably includes at least a single “reservoir” layer (i.e., a drug substrate) in combination with either at least a single limiting barrier, or a facilitating layer, or in combination with both at least a single limiting barrier and facilitating layer. By facilitating layer what is meant is a layer which may change its physical structure in situ, e.g., as by hydration, thus altering the capacity of the reservoir to elute a therapy agent (e.g., a facilitating layer underlying a reservoir layer may swell and act as a pressure pump to squeeze the reservoir layer against an outer barrier layer).

Generally, elements of the laminate, i.e., layers thereof, are advantageously comprised of urethanes, however, it is to be understood that layer composition may be readily adapted to accommodate more or less lipophilic agents as the therapy warrants. It is further, optionally contemplated that inorganic phases or pre-existing phases such as mico/macrospheres, on the order of about 10-200 microns, be provided integral to the structure, for example, as by encapsulation between and/or among select layers thereof. It is further noted that the porosity of the subject structure may be selectively controlled via the inclusion of phase separating solutions, preferably those characterized by swift evaporation.

As to structure fabrication, the layers thereof may be applied by dipping, spraying or layering pre-made sheets into tubular structures. These layers may be composed of any of solvent cast able polymeric materials including, polyvinylclorides, polyethlyacrylates, polyvinylnitriles and preferably polyurethanes as well as curable materials as water based latexes.

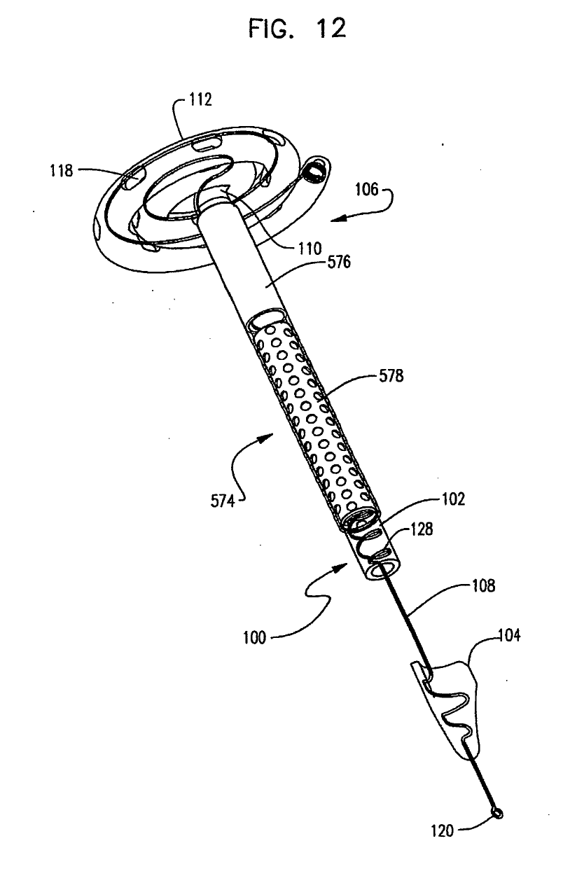

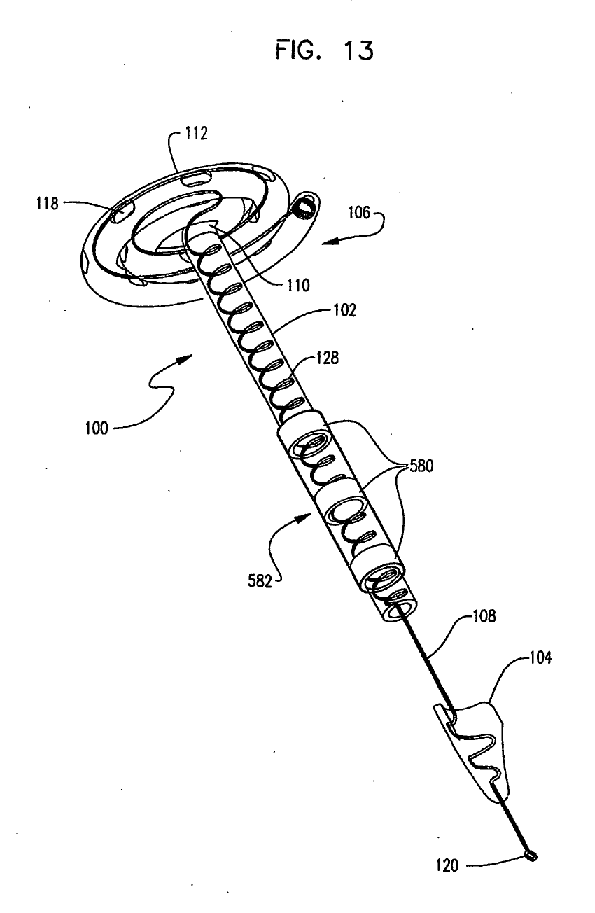

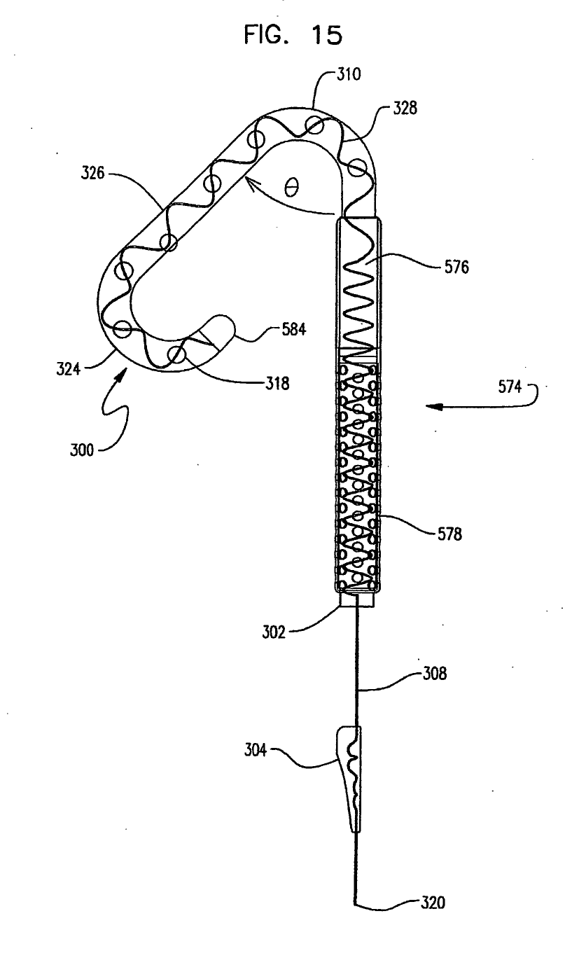

As to the illustrated structures, a “rolling” laminate sleeve 570, incorporating medicament carriers, e.g., bead-like elements 572, is shown in FIG. 11. A two component system is illustrated in FIG. 12, namely, a body conforming sleeve 574 having first 576 and second 578 zones for dedicated medicament storage/release functionality. It should be appreciated that the subject structure may selectively include a barrier substrate to effectuate a preselect medicament elution rate or quantity. Radioactive “brachytherapy” bands 580 are incorporated into a collar 582 of the structure of FIG. 13. In furtherance of the brachytherapy approach, a/k/a interstitial radiation or intra-cavitary radiation, the bands are constructed of a variety of moderately intense radioactive materials such as Iodine 125 which have short half-lives. A “solution” bulb 584, received within an aperture of the proximal end of the device, is finally illustrated in FIG. 14, with a combination of the bulb 584 and two component system of FIG. 12 illustrated in FIG. 15.

There are other variations of this invention which will become obvious to those skilled in the art. It will be understood that this disclosure, in many respects, is only illustrative. Although the various aspects of the present invention have been described with respect to various preferred embodiments thereof, it will be understood that the invention is entitled to protection within the full scope of the appended claims.

What is claimed is:

1. An indwelling urethral device comprising a prostatic urethral stent body and a urethral anchoring element, said prostatic urethral stent body including a preconfigured end portion for anchored receipt within a bladder, said urethral anchoring element extending from said prostatic urethral stent body via a linkage.

2. The indwelling urethral device of claim 1 wherein said preconfigured end portion is pretensioned.

3. The indwelling urethral device of claim 2 wherein said linkage comprises a tensile member.

4. The indwelling urethral device of claim 2 wherein said linkage comprises a tether.

5. The indwelling urethral device of claim 2 wherein said urethral anchoring element is spaced apart from said prostatic urethral stent body.

6. The indwelling urethral device of claim 5 wherein said urethral anchoring element is non-fixedly spaced apart from said prostatic urethral stent body.

7. The indwelling urethral device of claim 5 wherein said urethral anchoring element is non-rigidly spaced apart from said prostatic urethral stent body.

8. The indwelling urethral device of claim 2 wherein commencement of said pretensioned end portion of said prostatic urethral stent body is delimited by a bend of at least about 60° in said stent body.

9. The indwelling urethral device of claim 7 wherein said pretensioned end portion of said prostatic urethral stent body includes a free end segment.

10. The indwelling urethral device of claim 9 wherein said free end segment is characterized by a radial sweep.

11. The indwelling urethral device of claim 9 wherein said free end segment is characterized by a radial bend.

12. The indwelling urethral device of claim 8 wherein said pretensioned end portion of said prostatic urethral stent body includes a curved free end segment.

13. The indwelling urethral device of claim 12 wherein said curved free end comprises a hook.

14. The indwelling urethral device of claim 13 wherein said hook is spaced apart from said bend.

15. The indwelling urethral device of claim 13 wherein said hook is linearly spaced apart from said bend.

16. The indwelling urethral device of claim 2 wherein said pretensioned end portion of said prostatic urethral stent body includes a hooked free end segment.

17. The indwelling urethral device of claim 16 wherein said pretensioned end portion of said prostatic urethral stent body includes a urine ingress passage.

18. The indwelling urethral device of claim 17 wherein commencement of said pretensioned end portion of said prostatic urethral stent body is delimited by a bend of at least about 60° in said prostatic urethral stent body.

19. The indwelling urethral device of claim 18 wherein said pretensioned end portion of said prostatic urethral stent body comprises a spiraled end segment.

20. The indwelling urethral device of claim 18 wherein said pretensioned end portion of said prostatic urethral stent body comprises a rolled up free end.

21. The indwelling urethral device of claim 20 wherein said rolled up free end is adjacent said bend.

22. The indwelling urethral device of claim 20 wherein said rolled up free end forms a disc-like a bladder anchor.

23. The indwelling urethral device of claim 22 wherein said disc-like anchor has a lateral extent substantially perpendicular to an axis of elongation of said prostatic urethral stent body.

24. The indwelling urethral device of claim 22 wherein said disc-like anchor has a lateral extent substantially parallel to an axis of elongation of said prostatic urethral stent body.

25. The indwelling urethral device of claim 8 wherein said pretensioned end portion of said prostatic urethral stent body includes an integral reinforcing element.

26. The indwelling urethral device of claim 25 wherein said prostatic urethral stent body includes an integral reinforcing element.

27. The indwelling urethral device of claim 8 wherein said free end of said prostatic urethral stent body includes a structure adapted to retain and dispense a medicament.

28. The indwelling urethral device of claim 8 wherein said prostatic urethral stent body is adapted to retain and dispense a medicament.

29. The indwelling urethral device of claim 2 further comprising a structure adapted to retain and dispense a medicament.

30. The indwelling urethral device of claim 29 wherein said structure is carried by prostatic urethral stent body.

31. The indwelling urethral device of claim 30 wherein said structure is retained at a free end of said prostatic urethral stent body.

32. The indwelling urethral device of claim 30 wherein said structure is retained at a free end of said pretensioned end portion of said prostatic urethral stent body.

33. An endourethral device comprising a stent body, and proximal and distal anchors for retention of the device within a lower urinary tract, an anchor of said proximal and distal anchors being preconfigured so as to provide a self-adjusting functionality for the device.

34. A prostatic stent comprising a tubular element having a preconfigured free end portion and a substantially linear stent portion, said preconfigured free end portion having a bladder engaging segment, said bladder engaging segment being radially spaced apart from an axis of elongation of said stent portion.

35. An indwelling urethral device comprising a prostatic urethral stent body, a urethral anchoring element spaced apart therefrom, and an elongate tensile element extending through each of said prostatic urethral stent body and said urethral anchoring element, said prostatic urethral stent body including a preconfigured end portion for anchored receipt within a bladder, said elongate tensile element including first and second portions adjacent said urethral anchoring element.

36. A self adjusting endourethral device comprising an elongate tensile member, a device body reinforced by a proximal end portion of said elongate tensile member, a distal anchor spaced apart from said device body and supported by a distal end portion of said elongate tensile member, a free end of said elongate tensile member adapted to facilitate device retrieval, said device body including a pretensioned free end for proximally anchoring the device.

37. An endourethral stent comprising a stent body having proximal and distal portions and a distal anchor spaced apart from said distal end portion of said stent body, said stent body having an axis of elongation, said proximal portion of said stent body having a spiraled free end, said spiraled free end being adjacent said axis of elongation of said stent body.

38. An endourethral stent comprising a stent body having proximal and distal portions and a distal anchor spaced apart from said distal end portion of said stent body, said stent body having an axis of elongation, said proximal portion of said stent body having a spiraled free end wherein a spiral of said spiraled free end is characterized by an axis of rotation, said axis of elongation and said axis of rotation being substantially co-linear.

39. An endourethral stent comprising a stent body having proximal and distal portions and a distal anchor spaced apart from said distal end portion of said stent body, said proximal portion of said stent body comprising a linear segment terminating in an arcuate free end, said proximal portion of said stent body delimited by a stent body bend within a range of about 45-90°.

40. An endourethral stent comprising a stent body having proximal and distal portions, an elastic transition point delimiting said portions, and a distal anchor spaced apart from said distal end portion of said stent body, said proximal portion of said stent body comprising a hook-like free end spaced apart from said elastic transition point by a linear proximal stent body portion.
